(19) United States
(12) Patent Application Publication
(10) Pub. No.: US 2009/0156834 A1
(43) Pub. Date: Jun. 18, 2009

(54) PROCESS FOR MAKING 2,2-BIS (3,4-DICARBOXYPHENYL) HEXAFLUOROPROPANE

(75) Inventors: Andreas Kanschik-Conradsen, Garbsen (DE); Bjoern Oliver Jackisch, Isernhagen (DE); Ralph Lonsky, Neustadt (DE)

Correspondence Address: Honeywell International Inc.;Patent Services Department, 101 Columbia Road, Morristown, NJ, 07962, US

(73) Assignee: Honeywell International Inc.

(21) Appl. No.: 12/327,466

(22) Filed: Dec. 3, 2008

Related U.S. Application Data
(60) Provisional application No. 61/014,608, filed on Dec. 18, 2007.

(51) Int. Cl. C07D 407/10; C07C 51/275

(52) U.S. Cl. 549/241; 562/410

(57) ABSTRACT

There is provided a continuous process for making 2,2-bis(3,4-dicarboxyphenyl)hexafluoropropane. The process has the following steps: a) continuously feeding 2,2-bis(3,4-dimethylphenyl)hexafluoropropane and aqueous nitric acid into a reactor cell and b) heating the mixture to a temperature sufficient to effect reaction between the 2,2-bis(3,4-dimethylphenyl)hexafluoropropane and the nitric acid.

### CROSS-REFERENCE TO A RELATED APPLICATION

The present application claims priority from U.S. Provisional Application No. 61/014,608, filed Dec. 18, 2007, which is incorporated herein in its entirety.

### BACKGROUND OF THE INVENTION

1. Field of the Invention

The present invention relates to an continuous oxidation process for making 2,2-bis(3,4-dicarboxyphenyl)hexafluoropropane from 2,2-bis(3,4-dimethylphenyl)hexafluoropropane.

2. Description of the Prior Art

2,2-bis(3,4-dicarboxyphenyl)hexafluoropropane anhydride is a versatile compound used in making fluorinated polymers. Particularly, 2,2-bis(3,4-dicarboxyphenyl)hexafluoropropane anhydride can be reacted with diamines in polycondensation reactions to produce fluorinated polyimides. Such polyimides have distinct properties, including mechanical stability, resistance to chemicals, and heat resistance. These properties make fluorinated polyimides especially useful in gas separation applications. Some fluorinated polyimides also show low dielectric constants, qualifying them for certain applications in microelectronics. Additional, nonlimiting examples for the use of (fluorinated) polyimides can be found in Ullmann's Encyclopedia of Industrial Chemistry, vol. A21, pp. 253 (1992).

Preparation of fluorinated polyimides for the applications described above requires 2,2-bis(3,4-dicarboxyphenyl)hexafluoropropane anhydride of high purity. Various processes for preparing 2,2-bis(3,4-dicarboxyphenyl)hexafluoropropane anhydride by oxidation of 2,2-bis(3,4-dimethylphenyl)hexafluoropropane to 2,2-bis(3,4-dicarboxyphenyl)hexafluoropropane with subsequent formation of the anhydride have been described in the prior art. The oxidation of 2,2-bis(3,4-dimethylphenyl)hexafluoropropane to 2,2-bis(3,4-dicarboxyphenyl)hexafluoropropane impacts the purity of the 2,2-bis(3,4-dicarboxyphenyl)hexafluoropropane anhydride, as undesirable compounds may be introduced into the final product.

U.S. Pat. No. 3,310,573 describes a process for oxidizing 2,2-bis(3,4-dimethylphenyl)hexafluoropropane to 2,2-bis(3,4-dicarboxyphenyl)hexafluoropropane employing potassium permanganate as an oxidizing agent. However, the use of potassium permanganate as an oxidizing agent introduces a source for undesirable residual metal ions, especially manganese, in the reaction product, which would require further downstream purification.

European Patent Application No. 317,884 provides a process for oxidizing 2,2-bis(3,4-dimethylphenyl)hexafluoropropane to 2,2-bis(3,4-dicarboxyphenyl)hexafluoropropane employing catalytic air oxidation. In this process, cobalt, manganese and bromine are used as catalysts, which necessitates downstream purification of the oxidation product of the reaction.

U.S. Pat. No. 3,356,648 provides yet another process for oxidizing 2,2-bis(3,4-dimethylphenyl)hexafluoropropane to 2,2-bis(3,4-dicarboxyphenyl)hexafluoropropane. In this process, oxidation is done via autoclave nitric acid batch oxidation. This process is comparably advantageous over the two processes mentioned since no metal compounds are introduced. However, vigorous NOₓ formation in the course of the reaction, depending on the amounts of 2,2-bis(3,4-dimethylphenyl)hexafluoropropane and nitric acid present in the process, is a safety issues due to increased pressure. The safety issue is balanced with the need for economically reasonable batch sizes.

It would be desirable to have a process for making 2,2-bis(3,4-dicarboxyphenyl)hexafluoropropane from 2,2-bis(3,4-dimethylphenyl)hexafluoropropane in which no undesirable compounds, such as metal ions and/or halides, are introduced. It would further be desirable to have a process in which use as few as possible reactive compounds for safety reasons. It would be yet more desirable to have a process that would also allow for an economically reasonable throughput rate.

### SUMMARY OF THE INVENTION

According to the present invention, there is provided a process for making 2,2-bis(3,4-dicarboxyphenyl)hexafluoropropane. The process has the following steps: a) continuously feeding the reaction compounds 2,2-bis(3,4-dimethylphenyl)hexafluoropropane and nitric acid into a reactor cell and b) heating reaction compounds to a temperature sufficient to effect reaction between the 2,2-bis(3,4-dimethylphenyl)hexafluoropropane and the nitric acid to form 2,2-bis(3,4-dicarboxyphenyl)hexafluoropropane.

### DETAILED DESCRIPTION OF THE INVENTION

The process of the present invention employs oxidation to continuously convert 2,2-bis(3,4-dimethylphenyl)hexafluoropropane to 2,2-bis(3,4-dicarboxyphenyl)hexafluoropropane. The oxidizer is nitric acid (HNO₃). Reduced nitric acid can be reoxidized, e.g., via oxygenation, and can be used again in the reaction.

The oxidation reaction is the following:

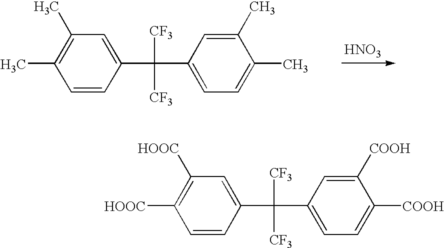

In a first step in the process of the present invention, 2,2-bis(3,4-dimethylphenyl)hexafluoropropane and nitric acid are fed continuously in a reactor cell.

Nitric acid is preferably employed at concentration of about 5% to about 95%, more preferably about 15% to about 65%, and most preferably about 25% to about 40%. The molar ratio of nitric acid to 2,2-bis(3,4-dimethylphenyl)hexafluoropropane in the mixture is preferably but not limited to about 4 to about 20, more preferably about 6 to about 12, and most preferably about 7 to about 10 by mole. Excess nitric acid can be recycled and used in the same process, thereby benefiting the environment due to low NO_{X}—waste. The above-recited molar ratio ranges for excess nitric acid are based substantially on economics rather than technical considerations. Thus, even higher molar ratios are possible.

In a second step in the process of the present invention, the mixture or slurry of 2,2-bis(3,4-dimethylphenyl)hexafluoropropane and nitric acid is heated in a reactor cell, where the two components are reacted via oxidation to form 2,2-bis(3,4-dicarboxyphenyl)hexafluoropropane.

The oxidation reaction takes place at a temperature and pressure sufficient to effect reaction. The reaction preferably takes place at a temperature from about 100° C. to about 300° C., more preferably about 180° C. to about 250° C., and most preferably about 200° C. to about 240° C.

The present invention affords high yields, i.e., high conversion levels of 2,2-bis(3,4-dimethylphenyl)hexafluoropropane. The conversion level is preferably about 90 mole % or more, more preferably about 93 mole % or more, and most preferably about 95 mole % (mole percent) or more.

Preferably, no undesirable compounds, such as metal ions and/or halides, are introduced into the process. Further preferably, the process uses as few reactive compounds as possible present in the system at any time of the reaction for safety reasons and is run continuously to reach economically reasonable throughput rates.

The reaction preferably takes place at a pressure from about 1,000 hectopascal (hPa) to about 85,000 hPa, more preferably about 10,000 hPa to about 40,000 hPa, and most preferably about 16,000 hPa to about 34,000 hPa.

The reaction cell useful in the process of the present invention refers to any reactor in which the two components are fed to the reactor, reacted, and moved therethrough in the nature of a plug pushed forward via backpressure from the continuous feed into the reactor. A preferred reaction cell is of a tubular reactor type, alternately referred to as a plug flow reactor. Such a reactor operates continuously, whereby the mixture or slurry entering the reactor will preferably have a residence time therein of about 1 minute (min.) to about 200 min., more preferably about 5 min. to about 50 min., and most preferably of about 10 min. to about 20 min.

The process of the present invention may also carried out in a microreactor. The microreactor is a microsystem having relatively small reaction dimensions (e.g. a typical diameter below 2 mm) and relatively high heat and mass transfer rates. The microreactor allows for precise adjustment of process conditions, short response times, and defined residence times, resulting in greater process control and higher yields and selectivity.

The process of the present invention may also carried out in a static mixer reactor. The advantages of a static mixer are comparable to the ones described above for tube reactors and microreactors, respectively.

The following examples of the process of the present invention are illustrative and not to be construed as limiting.

### EXAMPLE 1

2,2-bis(3,4-dimethylphenyl)hexafluoropropane is oxidized to 2,2-bis(3,4-dicarboxyphenyl)hexafluoropropane. 2,2-bis(3,4-dimethylphenyl)hexafluoropropane and 35% HNO₃ are fed into a continuous-flow tubular reactor. The tube diameter is 100 mm and length is 12 m. The temperature of the mixture within the reactor is increased to between 200° C. and 240° C. The volumetric flow in the reactor is set to 5.9 liters/minute. Nitric oxides are formed as a reaction by-product and are recycled to form HNO₃. The effluent from the reactor is collected, depressurized, and cooled to room temperature. HNO₃ is removed from the effluent. The desired product 2,2-bis(3,4-dicarboxyphenyl)hexafluoropropane precipitates within the effluent and is separated by centrifugation and washed with water. The purity of the product is 97% 2,2-bis(3,4-dicarboxyphenyl)hexafluoropropane as determined by high pressure liquid chromatography (HPLC).

### EXAMPLE 2

A volume flow of 100 ml/h of a mixture of 20 ml/h 2,2-bis(3,4-dimethylphenyl)hexafluoropropane and 70 ml/h 35% aqueous HNO₃ are fed into a microreactor. The micro reactor has of a micro mixer at the inlet having a typical diameter of about 2 mm and a volume of about 12 ml. The reaction pressure is controlled by a pressure control valve. The residence time part is divided into different sections (typically up to 4), which have a further feeding point with a consequent mixer for additionally fed HNO₃. Furthermore, each section can be heated individually to control the temperature profile over the residence time. The temperature along the residence time was 210° C., the pressure was about 19,000 hPa. In this example, no further HNO₃ was added in the feeding points. The reaction mixture is pumped through the reactor with a residence time of about 7 minutes leading to complete conversion and a selectivity of about 97%. The outflow of the reactor is depressurized and cooled to room temperature.

In the foregoing example, improved control of reaction temperature concentration allowed for improvement in selectivity by avoiding consecutive or side reactions compared to known processes.

### Formation of 2,2-bis(3,4-dicarboxyphenyl)hexafluoropropane anhydride

The anhydride formation reaction is represented by the following:

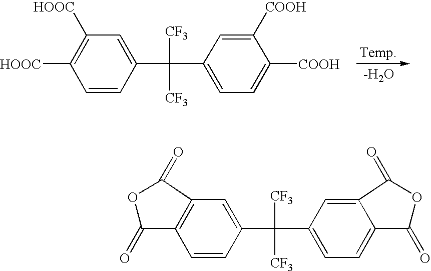

The 2,2-bis(3,4-dicarboxyphenyl)hexafluoropropane is subsequently converted to 2,2-bis(3,4-dicarboxyphenyl)hexafluoropropane anhydride. The 2,2-bis(3,4-dicarboxyphenyl)hexafluoropropane (water moist) is added to a hydrocarbon fraction having a boiling point of between 180° C. and 200° C. Residual water is distilled off. The mixture is further heated to 160° C. and reacted for 3 hours. The mixture is cooled to 20° C., and the 2,2-bis(3,4-dicarboxyphenyl)hexafluoropropane anhydride is separated via centrifugation, washed with acetic acid, and dried. The purity of the product is 97% 2,2-bis(3,4-dicarboxyphenyl)hexafluoropropane anhydride (HPLC). Recrystallization with acetic acid/acetanhydride/activated carbon yields a product 2,2-bis(3,4-dicarboxyphenyl)hexafluoropropane anhydride of >99% purity (HPLC).

It should be understood that the foregoing description is only illustrative of the present invention. Various alternatives and modifications can be devised by those skilled in the art without departing from the invention. Accordingly, the present invention is intended to embrace all such alternatives, modifications and variances which fall within the scope of the appended claims.

What is claimed is:

1. A continuous process for making 2,2-bis(3,4-dicarboxyphenyl)hexafluoropropane, comprising:
a) continuously feeding the reaction components into a reaction cell and
b) heating the mixture to a temperature sufficient to effect reaction between the 2,2-bis(3,4-dimethylphenyl)hexafluoropropane and the nitric acid.

2. The process of claim 1, wherein the nitric acid is about 5% to about 95%.

3. The process of claim 1, wherein the nitric acid is about 15% to about 65%.

4. The process of claim 1, wherein the nitric acid is about 25% to about 40%.

5. The process of claim 1, wherein the 2,2-bis(3,4-dimethylphenyl)hexafluoropropane and the nitric acid are reacted at about 100° C. to about 300° C.

6. The process of claim 1, wherein the 2,2-bis(3,4-dimethylphenyl)hexafluoropropane and the nitric acid are reacted at about 180° C. to about 250° C.

7. The process of claim 1, wherein the 2,2-bis(3,4-dimethylphenyl)hexafluoropropane and the nitric acid are reacted at about 200° C. to about 240° C.

8. The process of claim 1, wherein the ratio of nitric acid to 2,2-bis(3,4-dimethylphenyl)hexafluoropropane is about 4 to about 20 by mole.

9. The process of claim 1, wherein the ratio of nitric acid to 2,2-bis(3,4-dimethylphenyl)hexafluoropropane is about 6 to about 12 by mole.

10. The process of claim 1, wherein the ratio of nitric acid to 2,2-bis(3,4-dimethylphenyl)hexafluoropropane is about 7 to about 10 by mole.

11. The process of claim 1, wherein the retention time of the slurry in the reactor is about 1 min. to about 200 min.

12. The process of claim 1, wherein the retention time of the slurry in the reactor is about 3 min. to about 50 min.

13. The process of claim 1, wherein the retention time of the slurry in the reactor is about 5 min. to about 20 min.

14. The process of claim 1, wherein the conversion level is about 80 mole % or more.

15. The process of claim 1, wherein the conversion level is about 90 mole % or more.

16. The process of claim 1, wherein the conversion level is about 95 mole % or more.

17. The process of claim 1, further comprising coverting the 2,2-bis(3,4-dicarboxyphenyl)hexafluoropropane into 2,2-bis(3,4-dicarboxyphenyl)hexafluoropropane anhydride.
